(19) United States
(12) Patent Application Publication
(10) Pub. No.: US 2004/0033959 A1
(43) Pub. Date: Feb. 19, 2004

(54) Pharmaceutical compositions for hepatitis C viral protease inhibitors

(75) Inventors: Shirlynn Chen, Somers, NY (US); Xiaohui Mei, Highland Mills, NY (US)

Correspondence Address: BOEHRINGER INGELHEIM CORPORATION, 900 RIDGEBURY ROAD, P O BOX 368, RIDGEFIELD, CT, 06877, US

(73) Assignee: Boehringer Ingelheim Pharmaceuticals, Inc., Ridgefield, CT (US)

(21) Appl. No.: 10/620,408

(22) Filed: Jul. 16, 2003

Related U.S. Application Data
(60) Provisional application No. 60/397,280, filed on Jul. 19, 2002.

(51) Int. Cl. A61K 38/06

(52) U.S. Cl. 514/18

(57) ABSTRACT

Disclosed are pharmaceutical compositions of hepatitis C viral protease inhibitors having improved bioavailability, and methods of using these compositions for inhibiting the replication of the hepatitis C virus (HCV) and for the treatment of an HCV infection. These compositions include co-solvent systems, lipid based systems, solid dispersions and granulations, and all comprise the hepatitis C viral protease inhibitor, at least one pharmaceutically acceptable amine and optionally one or more additional ingredients.

### RELATED APPLICATIONS

This application claims the benefit of U.S. Provisional Application No. 60/397,280, filed Jul. 19, 2002, which application is herein incorporated by reference in its entirety.

### FIELD OF THE INVENTION

The present invention relates in general to pharmaceutical compositions of hepatitis C viral protease inhibitors having improved bioavailability, and methods of using these compositions for inhibiting the replication of the hepatitis C virus (HCV) and for the treatment of an HCV infection.

### BACKGROUND OF THE INVENTION

It has recently been discovered that certain peptide analogs are potent and specific inhibitors of hepatitis C virus (HCV) protease. In particular, compounds of the following formula I have been found to be an especially potent class of inhibitors against the NS3 serine protease of HCV:

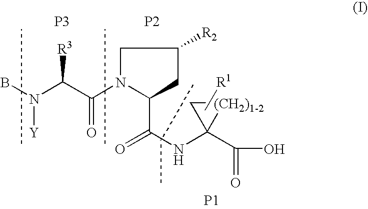

wherein B is H, a C₆ or C₁₀ aryl, C₇₋₁₆ aralkyl; Het or (lower alkyl)-Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide;

or B is an acyl derivative of formula R₄—C(O)—; a carboxyl derivative formula R₄—O—C(O)—; an amide derivative of formula R₄—N(R₅)—C(O)—; a thioamide derivative of formula R₄—N(R₅)—C(S)—; or a sulfonyl derivative of formula R₄—SO₂ wherein

R₄ is (i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;

(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;

(iii) amino optionally mono- or di-substituted with C₁₋₆ alkyl; amido; or (lower alkyl)amide;

(iv) C₆ or C₁₀ aryl or C₇₋₁₆ aralkyl, all optionally substituted with C₁₋₆ alkyl, hydroxy, amido, (lower alkyl)amide, or amino optionally mono- or di-substituted with C₁₋₆ alkyl; or

(v) Het or (lower alkyl)-Het, both optionally substituted with C₁₋₆ alkyl, hydroxy, amido, (lower alkyl) amide, or amino optionally mono- or di-substituted with C₁₋₆ alkyl;

R₅ is H or C₁₋₆ alkyl; with the proviso that when B is a carboxyl derivative, an amide derivative or a thioamide derivative, R₄ is not a cycloalkoxy;

Y is H or C₁₋₆ alkyl;

R³ is C₁₋₈ alkyl, C₃₋₇ cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;

R² is CH₂—R₂₀, NH—R₂₀, O—R₂₀ or S-R₂₀, wherein R₂₀ is quinolyl or (lower alkyl)quinolyl, both optionally mono-, di- or tri-substituted with R₂₁,

wherein each R₂₁ is independently C₁₋₆ alkyl; C₁₋₆ alkoxy; lower thioalkyl; sulfonyl; NO₂; OH; SH; halo; haloalkyl; amino optionally mono- or di-substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;

wherein R₂₂ is C₁₋₆ alkyl; C₃₋₇ cycloalkyl; C₁₋₆ alkoxy; amino optionally mono- or di-substituted with C₁₋₆ alkyl or C₃₋₇cycloalkyl; sulfonyl; (lower alkyl)sulfonyl; NO₂; OH; SH; halo; haloalkyl; carboxyl; amide; (lower alkyl)amide; or Het optionally substituted with C₁₋₆ alkyl;

R¹ is H; C₁₋₆ alkyl, C₃₋₇ cycloalkyl, C₂₋₆ alkenyl, or C₂₋₆ alkynyl, all optionally substituted with halogen;

or a tautomer thereof.

See, e.g., Llinas-Brunet et al., U.S. Pat. No. 6,323,180 B1, which is herein incorporated by reference in its entirety.

An HCV serine protease inhibitor such as the compounds of formula I would be expected to be an antiviral agent acting via a novel mechanism, i.e. blockage of a virus-encoded essential function for HCV replication. A drug acting through this mechanism should suppress viral replication of all HCV genotypes and therefore provide tangible benefits to patients with chronic hepatitis C.

A common problem among protease inhibitors is that these compounds are lipophilic and have low aqueous solubility. Because of the poor aqueous solubility, conventional solid and liquid pharmaceutical preparations containing these inhibitors may not be absorbed by the patient in a satisfactory manner. Of the various factors that can affect the bioavailability of a drug when administered orally, (which include aqueous solubility, drug absorption through the gastrointestinal tract, dosage strength and first pass effect), aqueous solubility is often found to be among the most important factors. Poorly water soluble compounds often exhibit either erratic or incomplete absorption in the digestive tract, and thus produce a less than desirable response.

Representative compounds of formula I have shown poor bioavailability when administered to animals, suggesting that conventional formulations containing these inhibitors may not be absorbed in a satisfactory manner. Thus, there is a need in the art for pharmaceutical compositions of the formula I compounds having improved bioavailability.

Examples of “self-emulsifying” formulations of lipophilic compounds include Lipari et al, WO 96/36316, which discloses a self-emulsifying pre-concentrate comprising a lipophilic compound, d-alpha-tocopheryl polyethylene glycol 1000 succinate (TPGS) and a lipophilic phase. Gao et al., U.S. Pat. No. 6,121,313 discloses a self-emulsifying formulation of a pyranone protease inhibitor comprising the pyranone compound, a mixture of mono- and di-glycerides, one or more solvents and one or more surfactants; and Gao et al, U.S. Pat. No. 6,231,887 B1 discloses a self-emulsifying formulation of a pyranone protease inhibitor comprising the pyranone compound, an amine, one or more solvents and one or more surfactants.

Yu et. al U.S. Pat. Nos. 5,360,615 and 5,071,643 disclose the preparation of a solvent system for enhancing the solubility of acidic, basic or amphoteric compounds by partial ionization comprising a mixture of polyethylene glycol, hydroxide or hydrogen ion, and water. Morton et al U.S. Pat. No. 5,376,688 discloses solutions of acidic, basic or amphoteric pharmaceutical agents comprising the pharmaceutical agent, an ionic species and a solvent system. Bhagwat et. al U.S. Pat. No. 6,056,977 teaches the use of polysaccharide based matrix for sustained release of a sulfonylurea.

Despite these advances, there continues to be a need in the art for oral pharmaceutical compositions of the compounds of formula I having improved bioavailability.

### BRIEF SUMMARY OF THE INVENTION

The present invention overcomes the aforementioned problems by providing pharmaceutical compositions of the formula I compounds having improved bioavailability as compared to conventional pharmaceutical formulations.

The pharmaceutical compositions of the present invention cover a wide variety of types of compositions, but all comprise a compound of formula I together with one or more pharmaceutically acceptable amines. The compositions of the present invention may include one or more additional ingredients depending on the type of composition contemplated, e.g., pharmaceutically acceptable bases, solvents, surfactants, oils, polymers, etc., as will be discussed in more detail below. The present invention is also directed to the methods of manufacturing these compositions, as described hereinafter.

In a general embodiment, the pharmaceutical composition of the present invention comprises:

(a) a compound of formula (I):

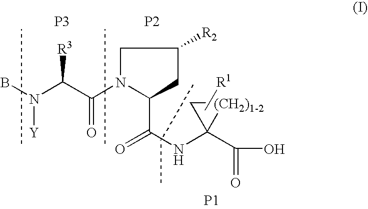

wherein B is H, a C₆ or C₁₀ aryl, C₇₋₁₆ aralkyl; Het or (lower alkyl)-Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide;

or B is an acyl derivative of formula R₄—C(O)—; a carboxyl derivative formula R₄—O—C(O)—; an amide derivative of formula R₄—N(R₅)—C(O)—; a thioamide derivative of formula R₄—N(R₅)—C(S)—; or a sulfonyl derivative of formula R₄—SO₂ wherein

R₄ is (i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;

(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;

(iii) amino optionally mono- or di-substituted with C₁₋₆ alkyl; amido; or (lower alkyl)amide;

(iv) C₆ or C₁₀ aryl or C₇₋₁₆ aralkyl, all optionally substituted with C₁₋₆ alkyl, hydroxy, amido, (lower alkyl)amide, or amino optionally mono- or di-substituted with C₁₋₆ alkyl; or

(v) Het or (lower alkyl)-Het, both optionally substituted with C₁₋₆ alkyl, hydroxy, amido, (lower alkyl) amide, or amino optionally mono- or di-substituted with C₁₋₆ alkyl;

R₅is H or C₁₋₆alkyl;

with the proviso that when B is a carboxyl derivative, an amide derivative or a thioamide derivative, R₄ is not a cycloalkoxy;

Y is H or C₁₋₆ alkyl;

R³ is C₁₋₈ alkyl, C₃₋₇ cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;

R² is CH₂—R₂₀, NH—R₂₀, O—R₂₀ or S—R₂₀, wherein R₂₀ is quinolyl or (lower alkyl)quinolyl, both optionally mono-, di- or tri-substituted with R₂₁,

wherein each R₂₁ is independently C₁₋₆ alkyl; C₁₋₆ alkoxy; lower thioalkyl; sulfonyl; NO₂; OH; SH; halo; haloalkyl; amino optionally mono- or di-substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;

wherein R₂₂ is C₁₋₆ alkyl; C₃₋₇ cycloalkyl; C₁₋₆ alkoxy; amino optionally mono- or di-substituted with C₁₋₆ alkyl or C₃₋₇cycloalkyl; sulfonyl; (lower alkyl)sulfonyl; NO₂; OH; SH; halo; haloalkyl; carboxyl; amide; (lower alkyl)amide; or Het optionally substituted with C₁₋₆ alkyl;

R¹ is H; C₁₋₆ alkyl, C₃₋₇ cycloalkyl, C₂₋₆ alkenyl, or C₂₋₆ alkynyl, all optionally substituted with halogen;

or a tautomer thereof;

(b) about 0.1 to 10% by weight of a pharmaceutically acceptable amine or a mixture of pharmaceutically acceptable amines; and

(c) one or more pharmaceutically acceptable oils, carriers or hydrophilic solvents; and when (c) is one or more pharmaceutically acceptable oils, the pharmaceutical composition further comprises:

(d) optionally one or more pharmaceutically acceptable hydrophilic solvents;

(e) optionally one or more pharmaceutically acceptable polymers; and

(f) optionally one or more pharmaceutically acceptable surfactants;

and when (c) is one or more pharmaceutically acceptable carriers, the pharmaceutical composition further comprises:

(d) optionally one or more pharmaceutically acceptable surfactants.

Another important aspect of the present invention involves a method of inhibiting the replication of hepatitis C virus by exposing the virus to a hepatitis C viral NS3 protease-inhibiting amount of a pharmaceutical composition of the present invention.

Another important aspect of the present invention involves a method of treating a hepatitis C viral infection in a mammal by administering to the mammal in need thereof a therapeutically effective amount of a pharmaceutical composition of the present invention.

### DETAILED DESCRIPTION OF THE INVENTION

### Definition of Terms and Conventions Used

Terms not specifically defined herein should be given the meanings that would be given to them by one of skill in the art in light of the disclosure and the context. As used in the specification, however, unless specified to the contrary, the following terms have the meaning indicated and the following conventions are adhered to.

A. Chemical and Pharmaceutical Nomenclature, Terms, and Conventions

In the groups, radicals, or moieties defined below, the number of carbon atoms is often specified preceding the group, for example, C₁₋₆ alkyl means an alkyl group or radical having 1 to 6 carbon atoms. In general, for groups comprising two or more subgroups, the last named group is the radical attachment point, for example, “thioalkyl” means a monovalent radical of the formula HS-Alk-. Unless otherwise specified below, conventional definitions of terms control and conventional stable atom valences are presumed and achieved in all formulas and groups.

The terms “amide” or “amido” as used herein, either alone or in combination with another substituent, mean a substituent of either of the following formulas: NH₂C(═O)— or HC(═O)NH—. The terms “susbtituted amide” and “substituted amido” mean either of the foregoing two groups wherein one or more of the hydrogen atoms are independently replaced.

The terms (lower alkyl)amide or (lower alkyl)amido as used herein, either alone or in combination with another substituent, mean an amide or amido group as defined above wherein one or more of the hydrogen atoms are independently replaced by a lower alkyl group.

The terms “C₁₋₆ alkyl” or “lower alkyl” as used herein, either alone or in combination with another substituent, means acyclic, straight or branched chain alkyl substituents containing from 1 to six carbon atoms and includes, for example, methyl, ethyl, propyl, butyl, hexyl, 1-methylethyl, 1-methylpropyl, 2-methylpropyl, and 1,1-dimethylethyl.

The term “C₃₋₆ cycloalkyl” as used herein, either alone or in combination with another substituent, means a cycloalkyl substituent containing from three to six carbon atoms and includes cyclopropyl, cyclobutyl, cyclopentyl, and cyclohexyl.

The terms “C₁₋₆ alkoxy” or “lower alkoxy” as used herein, either alone or in combination with another substituent, means the substituent C₁₋₆ alkyl-O— wherein alkyl is as defined above containing up to six carbon atoms. Alkoxy includes methoxy, ethoxy, propoxy, 1methylethoxy, butoxy and 1,1-dimethylethoxy. The latter substituent is known commonly as tert-butoxy.

The term “C₃₋₆ cycloalkoxy” as used herein, either alone or in combination with another substituent, means the substituent C₃₋₆ cycloalkyl-O— containing from 3 to 6 carbon atoms.

The term “halo” as used herein means a halogen substituent selected from bromo, chloro, fluoro or iodo.

The term “haloalkyl” as used herein means as used herein, either alone or in combination with another substituent, means acyclic, straight or branched chain alkyl substituents having one or more hydrogens substituted for a halogen selected from bromo, chloro, fluoro or iodo.

The term “thioalkyl” as used herein means as used herein, either alone or in combination with another substituent, means acyclic, straight or branched chain alkyl substituents containing a thiol (HS) group as a substituent. An example of a thioalkyl group is a thiopropyl, e.g., HS—CH₂CH₂CH₂— is one example of a thiopropyl group.

The term “C₁₋₆ or C₁₀ aryl” as used herein, either alone or in combination with another substituent, means either an aromatic monocyclic system containing 6 carbon atoms or an aromatic bicyclic system containing 10 carbon atoms. For example, aryl includes a phenyl or a naphthyl-ring system.

The term “Het” as used herein, either alone or in combination with another substituent, means a monovalent substituent derived by removal of a hydrogen from a five-, six-, or seven-membered saturated or unsaturated (including aromatic) heterocycle containing carbon atoms and from one to four ring heteroatoms selected from nitrogen, oxygen and sulfur. Examples of suitable heterocycles include: tetrahydrofuran, thiophene, diazepine, isoxazole, piperidine, dioxane, morpholine, pyrimidine or

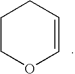

The term “Het” also includes a heterocycle as defined above fused to one or more other rings be it a heterocycle or any other cycle such as a benzene ring. One such examples includes thiazolo[4,5-b]-pyridine. Although generally covered under the term “Het”, the term “heteroaryl” as used herein precisely defines an unsaturated heterocycle for which the double bonds form an aromatic system. Suitable example of heteroaromatic system include: quinoline, indole, pyridine,

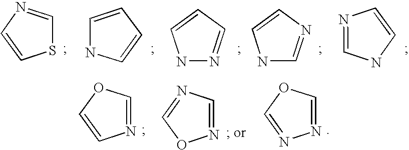

The term “oxo” means the double-bonded group (═O) attached as a substituent.

The term “thio” means the double-bonded group (═S) attached as a substituent.

The term “compounds of the invention”, and equivalent expressions, are meant to embrace compounds of Formula (I) as herein described, including the tautomers and isomers thereof, where the context so permits. In general, the compounds of the invention and the formulas designating the compounds of the invention are understood to only include the stable compounds thereof and exclude unstable compounds, even if an unstable compound might be considered to be literally embraced by the compound formula.

The term “stable compound” means a compound that is sufficiently robust to survive isolation to a useful degree of purity from a reaction mixture and formulation into an efficacious pharmaceutical composition. For example, a compound which would have a “dangling valency” or is a “carbanion” is not a compound contemplated by the invention.

The term “pharmaceutical composition of the invention” and equivalent expressions is meant to embrace all the various types of pharmaceutical compositions as described hereinafter, unless it is clear from the context that reference is being made to a particular type of pharmaceutical composition within the scope of the present invention.

The term “pharmaceutically acceptable” with respect to a substance as used herein means that substance which is, within the scope of sound medical judgment, suitable for use in contact with the tissues of humans and lower animals without undue toxicity, irritation, allergic response, and the like, commensurate with a reasonable benefit/risk ratio, and effective for the intended use when the substance is used in a pharmaceutical composition.

The term “semi-solid” means a material that is neither solid (elastic behavior) nor liquid (viscous behavior) and possesses the characteristics of both viscosity and elasticity. Examples of semi-solid materials include gels, ointments, creams, and highly viscous liquids.

The term “about” means within 20%, preferably within 10%, and more preferably within 5% of a given value or range. For example, “about 10%” means from 8% to 12%, preferably from 9% to 11%, and more preferably from 9.5% to 10.5%. When the term “about” is associated with a range of values, e.g., “about X to Y %”, the term “about” is intended to modify both the lower (X) and upper (Y) values of the recited range. For example, “about 0.1 to 10%” is equivalent to “about 0.1% to about 10%”. All percentages recited for amounts of ingredients in the compositions are percentages by weight with respect to the whole composition.

B. Isomer Terms and Conventions

The terms “isomers” or “stereoisomers” mean compounds having the same number and kind of atoms, and hence the same molecular weight, but differing in respect to the arrangement or configuration of the atoms in space. The term includes optical isomers and geometric isomers.

The term “optical isomer” means a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of formula I which may give rise to optical isomerism, the invention contemplates optical isomers and mixtures thereof. The compounds of formula I include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure optical isomers, i.e., as individual enantiomers or diastereomers, or as stercoisomer-enriched mixtures. Individual stereoisomers of compounds are prepared by synthesis from optically active starting materials containing the desired chiral centers or by preparation of mixtures of enantiomeric products followed by separation, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

The term “enantiomers” means a pair of optical isomers that are non-superimposable mirror images of each other.

The term “diastereoisomers” means optical isomers which are not mirror images of each other.

The term “racemic mixture” means a mixture containing equal parts of individual enantiomers.

The term “non-racemic mixture” means a mixture containing unequal parts of individual enantiomers or stereoisomers.

The term “geometrical isomer” means a stable isomer which results from restricted freedom of rotation about double bonds (e.g., cis-2-butene and trans-2-butene) or in a cyclic structure (e.g., cis-1,3-dichlorocyclobutane and trans-1,3-dichlorocyclobutane). Because carbon-carbon double (olefinic) bonds, cyclic structures, and the like may be present in the compounds of formula I, the invention contemplates each of the various stable geometric isomers and mixtures thereof resulting from the arrangement of substituents around these double bonds and in these cyclic structures. The substituents and the isomers are designated using the cis/trans convention.

Some of the compounds of formula I can exist in more than one tautomeric form. As mentioned above, the compounds of formula I include all such tautomers.

In general, all tautomeric forms and isomeric forms and mixtures, whether individual geometric isomers or optical isomers or racemic or non-racemic mixtures of isomers, of a chemical structure or compound is intended, unless the specific stereochemistry or isomeric form is specifically indicated in the compound name or structure.

C. Pharmaceutical Administration and Treatment Terms and Conventions

The term “patient” includes both human and non-human mammals.

The term “therapeutically effective amount” means an amount of a compound according to the invention which, when administered to a patient in need thereof, is sufficient to effect treatment of a hepatitis C viral infection. Further guidance with respect to determining suitable dosage levels for such effective treatment may be found in the “Methods of Therapeutic Use” section below. Such a therapeutically effective amount can be determined routinely by one of ordinary skill in the art having regard to their own knowledge, the prior art, and this disclosure.

The terms “treating” or “treatment” mean the treatment of a hepatitis C viral infection in a patient, and include:

(i) preventing the hepatitis C viral infection from occurring in a patient, in particular, when such patient is predisposed to such disease-state but has not yet been diagnosed as having it;

(ii) inhibiting or ameliorating the hepatitis C viral infection, i.e., arresting or slowing its development; or

(iii) relieving the hepatitis C viral infection, i.e., causing regression or cure of the disease-state.

### Preferred Embodiments of the Invention

I. Co-Solvent System

A first embodiment which we refer to herein as the “co-solvent” system is directed to a pharmaceutical composition comprising:

(a) a compound of formula (I):

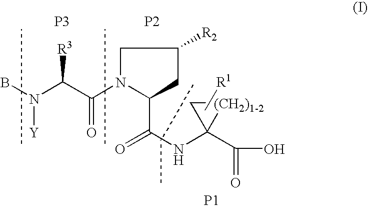

wherein B is H, a C₆ or C₁₀ aryl, C₇₋₁₆ aralkyl; Het or (lower alkyl)-Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide;

or B is an acyl derivative of formula R₄—C(O)—; a carboxyl derivative formula R₄—O—C(O)—; an amide derivative of formula R₄—N(R₅)—C(O)—; a thioamide derivative of formula R₄—N(R₅)—C(S)—; or a sulfonyl derivative of formula R₄—SO₂ wherein

R₄ is (i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;

(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;

(iii) amino optionally mono- or di-substituted with C₁₋₆ alkyl; amido; or (lower alkyl)amide;

(iv) C₆ or C₁₀ aryl or C₇₋₁₆ aralkyl, all optionally substituted with C₁₋₆ alkyl, hydroxy, amido, (lower alkyl)amide, or amino optionally mono- or di-substituted with C₁₋₆ alkyl; or

(v) Het or (lower alkyl)-Het, both optionally substituted with C₁₋₆ alkyl, hydroxy, amido, (lower alkyl) amide, or amino optionally mono- or di-substituted with C₁₋₆ alkyl;

R₅ is H or C₁₋₆ alkyl;

with the proviso that when B is a carboxyl derivative, an amide derivative or a thioamide derivative, R₄ is not a cycloalkoxy;

Y is H or C₁₋₆alkyl;

R³ is C₁₋₈ alkyl, C₃₋₇ cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;

R² is CH₂—R₂₀, NH—R₂₀, O—R₂₀ or S—R₂₀, wherein R₂₀ is quinolyl or (lower alkyl)quinolyl, both optionally mono-, di- or tri-substituted with R₂₁,

wherein each R₂₁ is independently C₁₋₆ alkyl; C₁₋₆ alkoxy; lower thioalkyl; sulfonyl; NO₂; OH; SH; halo; haloalkyl; amino optionally mono- or di-substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;

wherein R₂₂ is C₁₋₆ alkyl; C₃₋₇ cycloalkyl; C₁₋₆ alkoxy; amino optionally mono- or di-substituted with C₁₋₆ alkyl or C₃₋₇cycloalkyl; sulfonyl; (lower alkyl)sulfonyl; NO₂; OH; SH; halo; haloalkyl; carboxyl; amide; (lower alkyl)amide; or Het optionally substituted with C₁₋₆ alkyl;

R¹ is H; C₁₋₆ alkyl, C₃₋₇ cycloalkyl, C₂₋₆ alkenyl, or C₂₋₆ alkynyl, all optionally substituted with halogen;

or a tautomer thereof;

(b) about 0.1 to 10% by weight of a pharmaceutically acceptable amine or a mixture of pharmaceutically acceptable amines; and

(c) one or more pharmaceutically acceptable hydrophilic solvents.

The pharmaceutical composition may optionally further contain one or more pharmaceutically acceptable bases.

The amount of the active ingredient (formula (I) compound) that may be present in the co-solvent system composition may vary widely or be adjusted widely depending on the intended route of administration, the potency of the particular active ingredient being used, the severity of the hepatitis C viral infection and the required concentration. In a particular embodiment, the compound of formula (I) is present in the co-solvent system composition in an amount of from about 1% to 50% by weight, preferably from about 5% to 30% by weight, more preferably from about 5% to 15% by weight.

Pharmaceutically acceptable amines useful in the composition include, for example, C₁₋₆ alkylamine, di-(C₁₋₆ alkyl)-amine or tri-(C₁₋₆ alkyl)-amine, wherein one or more alkyl groups thereof may be optionally substituted by one or more hydroxy groups, or C₁₋₆ alkylenediamine, a basic amino acid or choline hydroxide, or mixtures thereof. Specific amines include ethanolamine, diethanolamine, triethanolamine, tris(hydroxymethyl)aminomethane, ethylenediamine or dimethylaminoethanol, or mixtures thereof A preferred amine is tris(hydroxymethyl)aminomethane (also called “Tris” or “Tromethamine”). The amine is present in an amount of about 0.1 to 10% by weight, more preferably in an amount of from about 0.5% to 7% by weight; even more preferably from about 0.5% to 5% by weight.

Pharmaceutically acceptable hydrophilic solvents useful in the composition include, for example, propylene glycol, polypropylene glycol, polyethylene glycol (e.g. PEG 400), glycerol, ethanol, dimethyl isosorbide, glycofurol, propylene carbonate, dimethyl acetamide, water, or mixtures thereof; preferably, propylene glycol, polyethylene glycol, ethanol, water, or mixtures thereof. A preferred solvent is a mixture of propylene glycol, ethanol and water. The amount of solvent(s) in the composition may vary over a wide range and the optimum amount for a particular composition will depend on the type and amount of other the other ingredients in the composition as can be easily determined by the skilled worker. In general, however, the solvent(s) are present in an amount of from about 40% to 99% by weight, preferably from about 80% to 99% by weight, more preferably, from about 80% to 90% by weight.

Pharmaceutically acceptable bases useful in the composition include, for example, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide. Also suitable are bases which are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Preferred cations include sodium, potassium, lithium, magnesium, calcium and ammonium. Some preferred bases include sodium hydroxide, potassium hydroxide, sodium hydrogen carbonate, aluminum hydroxide, magnesium hydroxide and magnesium aluminum hydroxide. When used in the composition, the pharmaceutically acceptable base is preferably present in the composition in an amount of from about 0.1 to 10% by weight, for example about 0.1 to 5% by weight, for example about 0.1 to 3% by weight.

A particular embodiment of the co-solvent system is directed to a pharmaceutical composition, comprising:

(a) about 5% to 30% by weight of a compound of formula (I);

(b) about 0.5% to 7% by weight of a pharmaceutically acceptable amine; and

(c) about 40% to 99% by weight of pharmaceutically acceptable hydrophilic solvent.

A further particular embodiment of the co-solvent system is directed to a pharmaceutical composition, comprising:

(a) about 5% to 15% by weight of a compound of formula (I);

(b) about 0.5% to 5% by weight of a pharmaceutically acceptable amine; and

(c) about 80% to 99% by weight of pharmaceutically acceptable hydrophilic solvent.

A further particular embodiment of the co-solvent system is directed to a pharmaceutical composition, comprising:

(a) about 5% to 15% by weight of a compound of formula (I);

(b) about 0.5% to 5% by weight of tris(hydroxymethyl)aminomethane; and

(c) about 80% to 90% by weight of a mixture of propylene glycol, ethanol and water.

The co-solvent system composition may be prepared in a conventional manner, for example, by dissolving the amine(s) in the pharmaceutically acceptable solvent(s), adding the compound of formula (I) to the resulting solution and then mixing the resulting solution until all or substantially all of the compound of formula I is solubilized in the solution. This method of preparing the composition constitutes another aspect of the present invention. The resulting solution is then formulated into the desired dosage form such as topical, parenteral and in particular oral dosage forms.

II. Lipid-Based System

A second embodiment which we refer to herein as the “Lipid-Based System” is directed to a pharmaceutical composition comprising:

(a) a compound of formula (I):

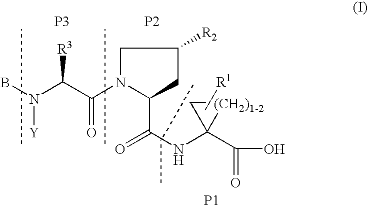

wherein B is H, a C₆ or C₁₀ aryl, C₇₋₁₆ aralkyl; Het or (lower alkyl)-Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide;

or B is an acyl derivative of formula R₄—C(O)—; a carboxyl derivative formula R₄—O—C(O)—; an amide derivative of formula R₄—N(R₅)—C(O)—; a thioamide derivative of formula R₄—N(R₅)—C(S)—; or a sulfonyl derivative of formula R₄—SO₂ wherein

R₄ is (i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;

(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;

(iii) amino optionally mono- or di-substituted with C₁₋₆ alkyl; amido; or (lower alkyl)amide;

(iv) C₆ or C₁₀ aryl or C₇₋₁₆ aralkyl, all optionally substituted with C₁₋₆ alkyl, hydroxy, amido, (lower alkyl)amide, or amino optionally mono- or di-substituted with C₁₋₆ alkyl; or

(v) Het or (lower alkyl)-Het, both optionally substituted with C₁₋₆ alkyl, hydroxy, amido, (lower alkyl) amide, or amino optionally mono- or di-substituted with C₁₋₆ alkyl;

R₅is H or C₁₋₆ alkyl;

with the proviso that when B is a carboxyl derivative, an amide derivative or a thioamide derivative, R₄ is not a cycloalkoxy;

Y is H or C₁₋₆ alkyl;

R³ is C₁₋₈ alkyl, C₃₋₇ cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;

R² is CH₂—R₂₀, NH—R₂₀, O—R₂₀ or S—R₂₀, wherein R₂₀ is quinolyl or (lower alkyl)quinolyl, both optionally mono-, di- or tri-substituted with R₂₁,

wherein each R₂₁ is independently C₁₋₆ alkyl; C₁₋₆ alkoxy; lower thioalkyl; sulfonyl; NO₂; OH; SH; halo; haloalkyl; amino optionally mono- or di-substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;

wherein R₂₂ is C₁₋₆ alkyl; C₃₋₇ cycloalkyl; C₁₋₆ alkoxy; amino optionally mono- or di-substituted with C₁₋₆ alkyl or C₃₋₇cycloalkyl; sulfonyl; (lower alkyl)sulfonyl; NO₂; OH; SH; halo; haloalkyl; carboxyl; amide; (lower alkyl)amide; or Het optionally substituted with C₁₋₆ alkyl;

R¹ is H; C₁₋₆ alkyl, C₃₋₇ cycloalkyl, C₂₋₆ alkenyl, or C₂₋₆ alkynyl, all optionally substituted with halogen;

or a tautomer thereof;

(b) about 0.1 to 10% by weight of a pharmaceutically acceptable amine or a mixture of pharmaceutically acceptable amines;

(c) one or more pharmaceutically acceptable oils;

(d) optionally one or more pharmaceutically acceptable hydrophilic solvents;

(e) optionally one or more pharmaceutically acceptable polymers; and

(f) optionally one or more pharmaceutically acceptable surfactants.

The composition may optionally further contain one or more pharmaceutically acceptable bases.

The amount of the active ingredient (formula (I) compound) that may be present in the lipid-based system composition may vary widely or be adjusted widely depending on the intended route of administration, the potency of the particular active ingredient being used, the severity of the hepatitis C viral infection and the required concentration. In a particular embodiment, the compound of formula (I) is present in the lipid-based system in an amount of from about 1% to 50% by weight, preferably from about 5% to 30% by weight, more preferably from about 10% to 20% by weight.

Pharmaceutically acceptable amines useful in the composition include, for example, C₁₋₆ alkylamine, di-(C₁₋₆ alkyl)-amine or tri-(C₁₋₆ alkyl)-amine, wherein one or more alkyl groups thereof may be optionally substituted by one or more hydroxy groups, or C₁₋₆ alkylenediamine, a basic amino acid or choline hydroxide, or mixtures thereof. Specific amines include ethanolamine, diethanolamine, triethanolamine, tris(hydroxymethyl)aminomethane, ethylenediamine or dimethylaminoethanol, or mixtures thereof. A preferred amine is tris(hydroxymethyl)aminomethane (also called “Tris”; and “tromethamine”). The amine is present in an amount of about 0.1 to 10% by weight, more preferably in an amount of from about 0.1% to 7% by weight; even more preferably from about 0.1% to 5% by weight.

Pharmaceutically acceptable bases useful in the composition include, for example, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide. Also suitable are bases which are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Preferred cations include sodium, potassium, lithium, magnesium, calcium and ammonium. Some preferred bases include sodium hydroxide, potassium hydroxide, sodium hydrogen carbonate, aluminum hydroxide, magnesium hydroxide and magnesium aluminum hydroxide. When used in the composition, the pharmaceutically acceptable base is present in the composition in an amount of from about 0.1 to 10% by weight, for example about 0.1 to 5% by weight, for example about 0.1 to 3% by weight.

Pharmaceutically acceptable oils useful in the composition includes a broad spectrum of water-immiscible materials such as, for example, medium or long chain mono-, di- or triglycerides, vegetable oils such as soybean oil, avocado oil, squalene oil, sesame oil, olive oil, canola oil, corn oil, rapeseed oil, safflower oil, and sunflower oil, fish oils, flavored oils, water insoluble vitamins, fatty acids, and mixtures thereof. More preferred oils include mono-, di- or triglycerides of caprylic fatty acids; mono-, di- or triglycerides of capric fatty acids; oleic acid, and mixtures thereof. Some preferred oils include those commercially available under the trade names: Capmul MCM, Capmul MCM C-8, Capmul MCM C-10, Capmul PG-8, Miglyol 810, Captex 355, Miglyol 812, Captex 200, Myvacet, Myverol 18-92, Maisine, and Arlacel 186. The amount of oil(s) in the composition may vary over a wide range and the optimum amount for a particular composition will depend on the type and amount of other the other ingredients in the composition as can be determined by the skilled pharmaceutical technician. In general, however, the pharmaceutically acceptable oil is present in an amount of from about 1% to 99% by weight, more preferably in an amount of from about 20% to 70% by weight.

In certain circumstances, e.g. for the purpose of increasing solubility, improving dispersability, pharmaceutically acceptable hydrophilic solvents can optionally be used in the composition, which include, for example, propylene glycol, polypropylene glycol, polyethylene glycol (e.g., PEG 400), glycerol, ethanol, dimethyl isosorbide, glycofurol, propylene carbonate, dimethyl acetamide, water, or mixtures thereof; preferably, propylene glycol, polyethylene glycol, ethanol, water, or mixtures thereof. A preferred solvent is a mixture of propylene glycol, ethanol and water. The amount of solvent in the composition may vary over a wide range and the optimum amount for a particular composition will depend on the type and amount of other the other ingredients in the composition as can be easily determined by the skilled worker. In general, however, the solvent(s) are present in an amount of up to about 70% by weight, preferably from about 10% to 30% by weight.

To adjust the viscosity of the formulations or to improve stability, pharmaceutically acceptable polymers can optionally be used in the composition, which include, for example, polyethylene glycols (e.g., PEG 1000, PEG 1500, PEG 3350, PEG 6000 and PEG 8000), polyvinylpyrrolidones (e.g., Kollidon 12 PF, Kollidon 17 PF, Kollidon 25 PF, Kollidon 30 PF, Kollidon 90 PF etc.), polyvinylalcohols, cellulose derivatives (e.g., hydroxypropylmethylcellulose (HPMC), hydroxypropylcellulose (HPC)), polyacrylates, polymethacrylates, sugars (e.g., lactose), polyols, and mixtures thereof. When used in the composition, the pharmaceutically acceptable polymer is preferably be present in an amount up to about 50% by weight, preferably about 1 to 20% by weight.

To facilitate self-emulsification, pharmaceutically acceptable surfactants can optionally be used in the composition, which include, for example, vitamin derivatives such as Vitamin E TPGS (d-alpha tocopheryl polyethylene glycol 1000 succinate), polyoxyl castor oils (e.g., Cremophor EL), polyoxyl hydrogenated castor oils, polysorbates (e.g., Tween 80), peglicol 6-oleate, polyoxyethylene stearates, polyglycolyzed glycerides (e.g., Gelucire 44/14) or poloxamers (e.g., Pluronic F68), sodium lauryl sulfate and mixtures thereof. Preferred surfactants include Vitamin E TPGS, polyoxyl 40 hydrogenated castor oil or polyoxyl 35 castor oil, and mixtures thereof.

When used in the composition, the surfactant is preferably present in an amount of up to about 70% by weight, preferably from about 20% to 50% by weight. This type of lipid-based system of the present invention further incorporating a surfactant is generally referred to herein as “self-emulsifying drug delivery system” or “SEDDS”.

A particular embodiment of the SEDDS composition according to the present invention is directed to a pharmaceutical composition, comprising:

(a) about 5% to 30% by weight of a compound of formula (I);

(b) about 0.1% to 7% by weight of a pharmaceutically acceptable amine;

(c) about 1% to 99% by weight of a pharmaceutically acceptable oil;

(d) up to about 70% by weight of a pharmaceutically acceptable hydrophilic solvent;

(e) optionally up to about 50% by weight of a pharmaceutically acceptable polymer;

(f) up to about 70% by weight of a pharmaceutically acceptable surfactant; and

(g) optionally about 0.1 to 10% by weight of a pharmaceutically acceptable base.

A further particular embodiment of the SEDDS composition according to the present invention is directed to a pharmaceutical composition, comprising:

(a) about 10% to 20% by weight of a compound of formula (I);

(b) about 0.1% to 5% by weight of a pharmaceutically acceptable amine;

(c) about 20% to 70% by weight of a pharmaceutically acceptable oil;

(d) about 10% to 30% by weight of a pharmaceutically acceptable hydrophilic solvent;

(e) optionally about 1% to 20% by weight of a pharmaceutically acceptable polymer; and

(f) about 20% to 50% by weight of a pharmaceutically acceptable surfactant; and;

(g) optionally about 0. 1 to 5% by weight of a pharmaceutically acceptable base.

A further particular embodiment of the SEDDS composition according to the present invention is directed to a pharmaceutical composition, comprising:

(a) about 10% to 20% by weight of a compound of formula (I);

(b) about 0.1% to 5% by weight of tris(hydroxymethyl)aminomethane;

(c) about 20% to 70% by weight of a mono- or diglyceride of caprylic fatty acid or a mono- or diglyceride of capric fatty acid, or mixtures thereof;

(d) about 10% to 30% by weight of a mixture of propylene glycol, ethanol and optionally water;

(e) optionally about 1% to 20% by weight of polyethylene glycol or polyvinylpyrrolidone; and

(f) about 20% to 50% by weight of d-alpha tocopheryl polyethylene glycol 1000 succinate or polyoxyl 35 castor oil (Cremophor EL); and

(g) optionally about 0.1 to 5% by weight of sodium hydroxide.

The Lipid-Based System composition may be prepared in a conventional manner, for example, by a method comprising: mixing together the liquid components, e.g., the pharmaceutically acceptable oil(s), and any surfactant(s) and solvent(s); dissolving the pharmaceutically acceptable amine(s) and polymer(s) in the resulting mixture; optionally heating the mixture obtained if necessary to sufficiently melt one or more of the components of the mixture; adding the compound of formula (I) to the resulting mixture and further mixing until all or substantially all of the compound of formula I is solubilized. This method of preparing the composition constitutes another aspect of the present invention. The resulting solution is then optionally formulated into the desired dosage form, for example, capsules, including hard shell or softgel capsules (e.g., hard or soft gelatin capsules), by known manufacturing technology. Examples of soft gelatin capsules that can be used include those disclosed in EP 649651 B1 and U.S. Pat. No. 5,985,321.

The composition may also be in the form of a liquid solution or semi-solid for oral, parenteral, rectal or topical administration.

III. Solid Dosage Forms

The present invention also contemplates and includes various solid dosage forms of the composition of the present invention, such as solid dispersions and granulations.

A. Solid Dispersions

The solid dispersion form of the composition of the present invention comprises:

(a) a compound of formula (I):

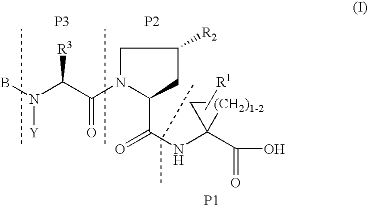

wherein B is H, a C₆ or C₁₀ aryl, C₇₋₁₆ aralkyl; Het or (lower alkyl)-Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide;

or B is an acyl derivative of formula R₄—C(O)—; a carboxyl derivative formula R₄—O—C(O)—; an amide derivative of formula R₄—N(R₅)—C(O)—; a thioamide derivative of formula R₄—N(R₅)—C(S)—; or a sulfonyl derivative of formula R₄—SO₂ wherein

R₄ is (i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;

(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;

(iii) amino optionally mono- or di-substituted with C₁₋₆ alkyl; amido; or (lower alkyl)amide;

(iv) C₆ or C₁₀ aryl or C₇₋₁₆ aralkyl, all optionally substituted with C₁₋₆ alkyl, hydroxy, amido, (lower alkyl)amide, or amino optionally mono- or di-substituted with C₁₋₆ alkyl; or

(v) Het or (lower alkyl)-Het, both optionally substituted with C₁₋₆ alkyl, hydroxy, amido, (lower alkyl) amide, or amino optionally mono- or di-substituted with C₁₋₆ alkyl;

R₅ is H or C₁₋₆ alkyl;

with the proviso that when B is a carboxyl derivative, an amide derivative or a thioamide derivative, R₄ is not a cycloalkoxy;

Y is H or C₁₋₆ alkyl;

R³ is C₁₋₈ alkyl, C3-7 cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;

R₂ is CH₂—R₂₀, NH—R₂₀, O—R₂₀ or S—R₂₀, wherein R₂₀ is quinolyl or (lower alkyl)quinolyl, both optionally mono-, di- or tri-substituted with R₂₁,

wherein each R₂₁ is independently C₁₋₆ alkyl; C₁₋₆ alkoxy; lower thioalkyl; sulfonyl; NO₂; OH; SH; halo; haloalkyl; amino optionally mono- or di-substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;

wherein R₂₂ is C₁₋₆ alkyl; C₃₋₇ cycloalkyl; C₁₋₆ alkoxy; amino optionally mono- or di-substituted with C₁₋₆ alkyl or C₃₋₇cycloalkyl; sulfonyl; (lower alkyl)sulfonyl; NO₂; OH; SH; halo; haloalkyl; carboxyl; amide; (lower alkyl)amide; or Het optionally substituted with C₁₋₆ alkyl;

R¹ is H; C₁₋₆ alkyl, C₃₋₇ cycloalkyl, C₂₋₆ alkenyl, or C₂₋₆ alkynyl, all optionally substituted with halogen;

or a tautomer thereof;

b) about 0.1 to 10% by weight of a pharmaceutically acceptable amine or a mixture of pharmaceutically acceptable amines;

c) one or more pharmaceutically acceptable carriers; and

d) optionally one or more pharmaceutically acceptable surfactants.

The pharmaceutical composition may optionally further contain one or more pharmaceutically acceptable bases.

The amount of the active ingredient (formula (I) compound) that may be present in the solid dispersion composition may vary widely or be adjusted widely depending on the intended route of administration, the potency of the particular active ingredient being used, the severity of the hepatitis C viral infection and the required concentration. In a particular embodiment, the compound of formula (I) is present in the solid dispersion in an amount of from about 1% to 50% by weight, preferably from about 5% to 30% by weight, more preferably from about 10% to 20% by weight.

Pharmaceutically acceptable amines useful in the composition include, for example, C₁₋₆ alkylamine, di-(C₁₋₆ alkyl)-amine or tri-(C₁₋₆ alkyl)-amine, wherein one or more alkyl groups thereof may be optionally substituted by one or more hydroxy groups, or C₁₋₆ alkylenediamine, a basic amino acid or choline hydroxide, or mixtures thereof. Specific amines include ethanolamine, diethanolamine, triethanolamine, tris(hydroxymethyl)aminomethane, ethylenediamine or dimethylaminoethanol, or mixtures thereof. A preferred amine is tris(hydroxymethyl)aminomethane (also called “Tris”; and “tromethamine”). The amine is present in an amount of about 0.1 to 10% by weight, more preferably in an amount of from about 0.1% to 7% by weight; even more preferably from about 0.1% to 5% by weight.

Pharmaceutically acceptable carriers that can be used in the composition include any substance that can effectively retain the active ingredient of formula (I) in dispersed state in a final solid dosage form. Suitable pharmaceutically acceptable carriers include, for example, pharmaceutically acceptable polymers and pharmaceutically acceptable ureas. Preferred carriers include polyethylene glycols (e.g., PEG 1000, PEG 1500, PEG 3350, PEG 4600, PEG 6000 and PEG 8000), polyvinylpyrrolidones (e.g., Kollidon 12 PF, Kollidon 17 PF, Kollidon 25 PF, Kollidon 30 PF, Kollidon 90 PF etc.), polyvinylalcohols, cellulose derivatives (e.g., hydroxypropylmethylcellulose (HPMC), hydroxypropylcellulose (HPC)), polyacrylates, polymethacrylates, polyglycolyzed glycerides, ureas, sugars (e.g., lactose), polyols, and mixtures thereof. The best carrier to be used for a particular composition will depend on a variety of factors including the other ingredients in the composition and the specific method to be employed in the preparation of the composition, e.g., co-melting or co-precipitation, as discussed below. For example, when preparing the composition using the co-melt process it is desirable to use a carrier that can be melted under suitable laboratory conditions, for example, at less than about 100° C., preferably less than about 80° C. When preparing the composition using the co-precipitation process it is desirable to use a carrier that can be dissolved in a suitable hydrophilic solvent along with the other ingredients such that co-precipitation can take place.

The amount of pharmaceutically acceptable carrier may vary over a wide range and the optimum amount for a particular composition will again depend on the other ingredients in the composition and the method of preparation to be employed, and can be easily determined by the skilled pharmaceutical technician. In general, however, the pharmaceutically acceptable carrier may be present in the solid dispersion composition in an amount up from about 1 to 99% by weight, preferably about 60% to 80% by weight.

In order to achieve improved dispersion and dissolution performance, pharmaceutically acceptable surfactants can optionally be used in the composition, which include, for example, vitamin derivatives such as Vitamin E TPGS (d-alpha tocopheryl polyethylene glycol 1000 succinate), polyoxyl castor oils (e.g., Cremophor EL), polyoxyl hydrogenated castor oils, polysorbates (e.g., Tween 80), peglicol 6-oleate, polyoxyethylene stearates, polyglycolyzed glycerides such as lauroyl macrogoglycerides (Gelucire 44/14), poloxamers such as polyoxypropylene-polyoxyethylene block copolymer (Pluronic F68), sodium lauryl sulfate (SLS) and mixtures thereof. Preferred surfactants include Vitamin E TPGS, Pluronic F68, or sodium lauryl sulfate, and mixtures thereof. When used in the composition, the surfactant is preferably present in an amount of up to about 50% by weight, preferably from about 1% to 20% by weight.

Pharmaceutically acceptable bases useful in the composition include, for example, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide. Also suitable are bases which are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Preferred cations include sodium, potassium, lithium, magnesium, calcium and ammonium. Some preferred bases include sodium hydroxide, potassium hydroxide, sodium hydrogen carbonate, aluminum hydroxide, magnesium hydroxide and magnesium aluminum hydroxide. When used in the composition, the pharmaceutically acceptable base is preferably present in the composition in an amount of from about 0.1 to 10% by weight, for example about 0.1 to 5% by weight, for example about 0.1 to 3% by weight.

A particular embodiment of the solid dispersion composition is directed to a pharmaceutical composition comprising:

(a) about 5% to 30% by weight of a compound of formula (I);

(b) about 0. 1% to 7% by weight of a pharmaceutically acceptable amine;

(c) about 1% to 99% by weight of a pharmaceutically acceptable carrier; and

(d) up to about 50% by weight of a pharmaceutically acceptable surfactant.

A further particular embodiment of the solid dispersion composition is directed to a pharmaceutical composition comprising:

(a) about 10% to 20% by weight of a compound of formula (I);

(b) about 0.1% to 5% by weight of a pharmaceutically acceptable amine;

(c) about 60% to 80% by weight of a pharmaceutically acceptable carrier; and

(d) about 1% to 20% by weight of a pharmaceutically acceptable surfactant.

A further particular embodiment of the solid dispersion composition is directed to a pharmaceutical composition comprising:

(a) about 10% to 20% by weight of a compound of formula (I);

(b) about 0.1% to 5% by weight of tris(hydroxymethyl)aminomethane;

(c) about 60% to 80% by weight of polyethylene glycol, polyvinylpyrrolidone, lactose or a mixture thereof; and

(d) about 1% to 20% by weight of d-alpha tocopheryl polyethylene glycol 1000 succinate, polyoxypropylene-polyoxyethylene block copolymer, or sodium lauryl sulfate.

The solid dispersion composition may be prepared by two alternative methods: the co-melt method or the co-precipitation method, each of which constitutes another aspect of the present invention.

The co-melt method comprises: (a) mixing the pharmaceutically acceptable carrier(s) and the optional surfactant(s) and heating the resulting mixture to sufficiently melt the carrier(s) and surfactant(s); (b) adding the pharmaceutically acceptable amine(s) and the compound of formula (I) to the mixture obtained in step (a) and mixing until all or substantially all of the compound of formula (I) is solubilized. The resulting dispersion is then allowed to cool and form a solid or semi-solid dispersion. The resulting dispersion is then optionally formulated into the desired dosage form such as, for example, capsules, including hard shell or softgel capsules, by known manufacturing technology. Examples of soft gelatin capsules that can be used include those disclosed in EP 649651 B1 and U.S. Pat. No. 5,985,321.

The co-precipitation method comprises: (a) dissolving the pharmaceutically acceptable amine(s), the pharmaceutically acceptable carrier(s) and optionally the pharmaceutically acceptable surfactant(s) in a suitable hydrophilic solvent; (b) adding the compound of formula (I) to the solution obtained in step (a) and mixing to dissolve the compound of formula (I); and (c) evaporating the hydrophilic solvent to cause co-precipitation of the compound of formula (I), the amine(s), the carrier(s) and the optional surfactant(s). Preferred hydrophilic solvents for use in this process include ethanol, methanol and chloroform. The resulting co-precipitated solid or semi-solid dispersion, generally a powder, is then optionally formulated into the desired dosage form such as, for example, tablets or capsules, including hard shell or softgel capsules, by known manufacturing technology. Examples of soft gelatin capsules that can be used include those disclosed in EP 649651 B1 and U.S. Pat. No. 5,985,321.

B. Granulations

The pharmaceutical compositions of the present invention may also be in the form of granulations which are prepared using conventional granulation techniques. Such granulations may generally comprise the same ingredients in the same amounts as is set forth above with respect to the solid dispersion compositions according to the present invention. The resulting granulation is then optionally formulated into the desired dosage form such as, for example, compressed into tablets or filled into capsules, including hard shell capsules, by known manufacturing technology.

The granulations may be prepared by two alternative methods: dry granulation method and wet granulation method, each of which constitutes another aspect of the present invention.

The dry granulation method comprises: (a) triturating and mixing the compound of formula (I), the pharmaceutically acceptable amine(s), the pharmaceutically acceptable carrier(s) and optionally the pharmaceutically acceptable surfactant(s) to form a blend; and (b) optionally adding to the blend a lubricant, e.g. <1% by weight of magnesium stearate. The resulting blended powder may be compressed into tablets.

The wet granulation method comprises: (a) mixing the compound of formula (I), the pharmaceutically acceptable amine(s), the pharmaceutically acceptable carrier(s) and optionally the pharmaceutically acceptable surfactant(s) while adding water or another hydrophilic solvent(s) to the mixture to obtain a paste; (b) drying the paste of step (a) to a sufficient level of dryness; and (c) passing the dried paste through a screen. The resulting granules may be filled into capsules or compressed into tablets.

IV. Optional Additional Ingredients

If desired, the compositions according to the present invention may further include conventional pharmaceutical additives as is necessary or desirable to obtain a suitable formulation, such as antioxidants, lubricants, disintegrants, preservatives, buffers, stabilizers, thickening agents, coloring agents, sweetening agents, flavoring agents, fragrances, etc. Additional additives that may be useful in the compositions of the invention are disclosed in Llinas-Brunet et al., U.S. Pat. No. 6,323,180 B 1.

In one preferred embodiment, the compositions according to the present invention further contain one or more antioxidants. Preferred antioxidants include, for example, ascorbic acid, sulfatide salts, citric acid, propyl gallate, dl-α-tocopherol, ascorbyl palmitate, BHT or BHA. If present, the antioxidant is generally present in an amount of from about 0.01% to 1% by weight.

V. Compounds of Formula (I)

More specific embodiments for the compounds of formula (I) in the compositions are as set forth below.

One embodiment is directed to a compound of formula (I) wherein:

B is a carboxyl derivative of formula R₄—O—C(O)—, wherein R₄ is

(i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido or (lower alkyl)amide;

(ii) C₃₋₇ cycloalkyl, C₄₋₁₀ alkylcycloalkyl, all optionally substituted with carboxyl, (C₁₋₆ alkoxy)carbonyl, amino optionally mono- or di-substituted with C₁₋₆ alkyl, amido or (lower alkyl)amide;

(iv) C₆ or C₁₀ aryl or C₇₋₁₆ aralkyl optionally substituted with C₁₋₆ alkyl, hydroxy, amido, (lower alkyl)amido, or amino optionally mono- or di-substituted with C₁₋₆ alkyl; or

(v) Het or (lower alkyl)-Het, both optionally substituted with C₁₋₆ alkyl, hydroxy, amino optionally mono- or di-substituted with C₁₋₆ alkyl, amido or (lower alkyl)amido.

Another embodiment is directed to a compound of formula (I) wherein:

B is a carboxyl derivative of formula R₄—O—C(O)—, wherein R₄ is

(i) C₁₋₁₀ alkyl optionally substituted with carboxyl, C₁₋₆ alkanoyl, hydroxy, C₁₋₆ alkoxy or amido, (lower alkyl)amide, amino optionally mono- or di-substituted with C₁₋₆ alkyl; or

(ii) C₃₋₇ cycloalkyl, C₄₋₁₀ alkylcycloalkyl, all optionally substituted with carboxyl, (C₁₋₆ alkoxy)carbonyl, amido, (lower alkyl)amide, amino optionally mono- or di-substituted with C₁₋₆ alkyl.

Another embodiment is directed to a compound of formula (I) wherein: Y is H or methyl.

Another embodiment is directed to a compound of formula (I) wherein:

R³ is C₁₋₈ alkyl, C₃₋₇ cycloalkyl, or C₄₋₁₀ alkylcycloalkyl, all optionally substituted with hydroxy, C₁₋₆ alkoxy, C₁₋₆ thioalkyl, acetamido, C₆ or C₁₀ aryl, or C₇₋₁₆ aralkyl.

Another embodiment is directed to a compound of formula (I) wherein: R³ is C₁₋₈ alkyl, for example, tert-butyl.

Another embodiment is directed to a compound of formula (I) wherein:

R₂₁, is C₁₋₆ alkyl; C₁₋₆ alkoxy; lower thioalkyl; amino or amido optionally mono-or di-substituted with C₁₋₆ alkyl, C₆ or C₁₀ aryl, C₇₋₁₆ aralkyl, Het or (lower alkyl)-Het; NO₂; OH; halo; trifluoromethyl; carboxyl; C₆ or C₁₀ aryl, C₇₋₁₆ aralkyl, or Het, said aryl, aralkyl or Het being optionally substituted with R₂₂, wherein

R₂₂ is C₁₋₆ alkyl; C₃₋₇ cycloalkyl; C₁₋₆ alkoxy; amino optionally mono- or di-substituted with C₁₋₆alkyl or C₃₋₇ cycloalkyl; amide; (lower alkyl)amide; sulfonylalkyl; NO₂; OH; halo; trifluoromethyl; carboxyl or Het.

Another embodiment is directed to a compound of formula (I) wherein:

R₂₁ is C₁₋₆ alkyl; C₁₋₆ alkoxy; amino; di(lower alkyl)amino; (lower alkyl)amide; C₆ or C₁₀ aryl, or Het, said aryl or Het being optionally substituted with R₂₂, wherein R₂₂ is C₁₋₆ alkyl; C₃₋₇ cycloalkyl; C₁₋₆ alkoxy; amino optionally mono- or di-substituted with C₁₋₆alkyl or C₃₋₇ cycloalkyl; amido; (lower alkyl)amide; halo; trifluoromethyl or Het.

Another embodiment is directed to a compound of formula (I) as described above wherein R₂₂ is amino optionally mono- or di-substituted with C₁₋₆alkyl or C₃₋₇ cycloalkyl; amido; or C₁₋₆ alkyl-C(O)—NH—.

Another embodiment is directed to a compound of formula (I) wherein R² is selected from the group consisting of:

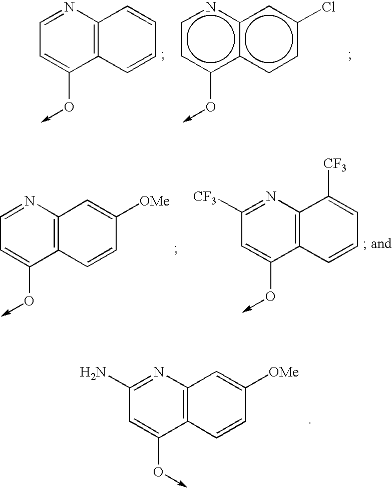

Another embodiment is directed to a compound of formula (I) wherein R₂ is:

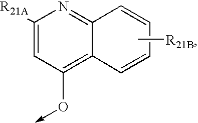

wherein R_{21A} is C₁₋₆ alkyl; C₁₋₆ alkoxy; lower thioalkyl; halo; amino optionally mono-substituted with C₁₋₆ alkyl; or C₆, C₁₀ aryl, C₇₋₁₆ aralkyl, or Het, said aryl, aralkyl or Het optionally substituted with R₂₂ wherein R₂₂ is C₁₋₆ alkyl; C₁₋₆ alkoxy; amido; amino optionally mono- or di-substituted with C₁₋₆alkyl or C₃₋₇ cycloalkyl; (lower alkyl)amide or Het; and

R_{21B} is C₁₋₆ alkyl, C₁₋₆ alkoxy, amino, di(lower alkyl)amino, (lower alkyl)amide, NO₂, OH, halo, trifluoromethyl, or carboxyl.

Another embodiment is directed to a compound of formula (I) as described above and wherein R_{21A} is C₆, C₁₀ aryl or Het, all optionally substituted with R₂₂ as defined above.

Another embodiment is directed to a compound of formula (I) as described above and wherein R_{21A} is selected from the group consisting of:

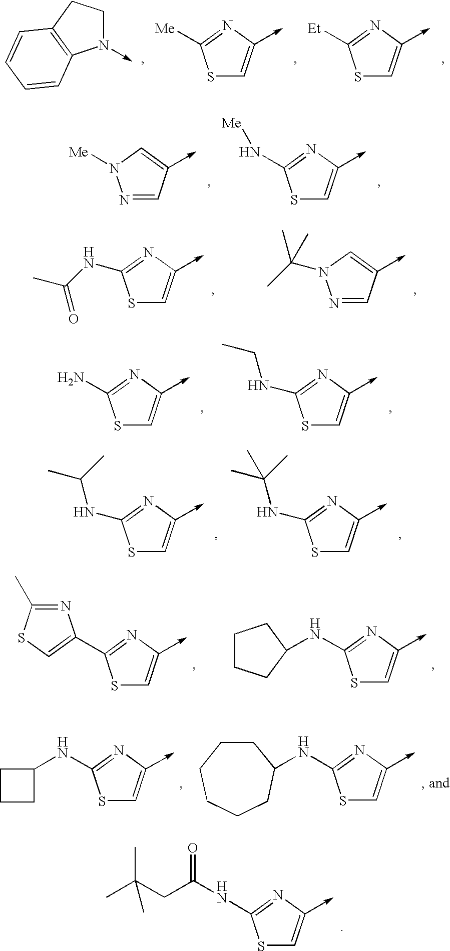

Another embodiment is directed to a compound of formula (I) wherein R² is:

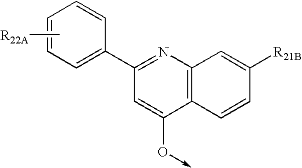

wherein R_{22A} is C₁₋₆ alkyl; C₁₋₆ alkoxy; or halo; and R_{21B} is C₁₋₆ alkyl, C₁₋₆ alkoxy, amino, di(lower alkyl)amino, (lower alkyl)amide, NO₂, OH, halo, trifluoromethyl, or carboxyl.

Another embodiment is directed to a compound of formula (I) wherein R² is:

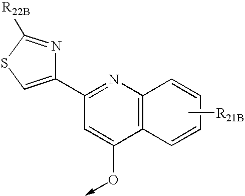

wherein R_{22B} is C₁₋₆ alkyl; amino optionally mono- or di-substituted with C₁₋₆alkyl or C₃₋₇ cycloalkyl; (lower alkyl)amide; or amido; and R_{21B} is C₁₋₆ alkyl, C₁₋₆ alkoxy, amino, di(lower alkyl)amino, (lower alkyl)amide, NO₂, OH, halo, trifluoromethyl, or carboxyl.

Another embodiment is directed to a compound of formula (I) as described above and wherein R_{21B} is C₁₋₆ alkoxy or di(lower alkyl)amino; and R_{22B} is amino mono-substituted with C₃₋₇ cycloalkyl; or is C₁₋₆ alkyl-C(O)—NH—.

Another embodiment is directed to a compound of formula (I) as described above and wherein wherein R_{21B} is methoxy or dimethylamino.

Another embodiment is directed to a compound of formula (I) as described above and wherein R¹ is H, C₁₋₃ alkyl, C₃₋₅ cycloalkyl, or C₂₋₄ alkenyl, all optionally substituted with halo.

Another embodiment is directed to a compound of formula (I) as described above and

wherein P1 is

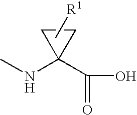

and R¹ is ethyl, vinyl, cyclopropyl, 1 or 2-bromoethyl or 1 or 2-bromovinyl, and more specifically wherein R¹ is vinyl.

When R¹ is not H, then in one embodiment P1 contains a cyclopropyl system of formula:

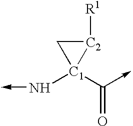

wherein C₁ and C₂ each represent an asymmetric carbon atom at positions 1 and 2 of the cyclopropyl ring. Notwithstanding other possible asymmetric centers at other segments of the compounds of formula I, the presence of these two asymmetric centers means that the compounds of formula I can exist as racemic mixtures of diastereoisomers. The racemic mixtures can be prepared and thereafter separated into individual optical isomers, or these optical isomers can be prepared by chiral synthesis, using conventional methods.

Hence, the compounds of formula I can exist as a racemic mixture of diastereoisomers at carbon 1 but wherein R¹ at carbon 2 is orientated syn to the carbonyl at position 1, represented by the radical:

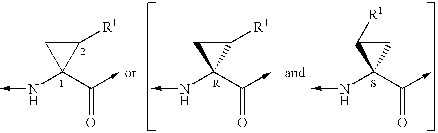

or the compound of formula I can exist as a racemic mixture of diastereoisomers wherein

R¹ at position 2 is orientated anti to the carbonyl at position 1, represented by the radical:

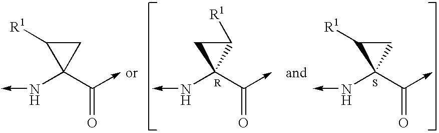

In turn, the racemic mixtures can be separated into individual optical isomers.

A particular embodiment is one wherein R¹ is not H and carbon 1 has the R configuration.

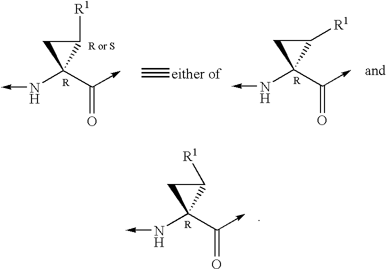

Another particular embodiment is one wherein R¹ at carbon 2 is orientated syn to the carbonyl at position 1, represented by the radical:

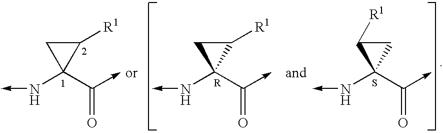

Another particular embodiment is one wherein said R¹ substituent and the carbonyl are in a syn orientation in the following absolute configuration:

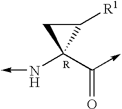

Another embodiment is directed to a compound of formula (I) as described above and wherein R¹ is ethyl.

Another embodiment is directed to a compound of formula (I) as described above and wherein R¹ is vinyl.

Another embodiment is directed to a compound of formula (I) wherein:

B is a carboxyl derivative of formula R₄—O—C(O)—, wherein R₄ is C₃₋₇ cycloalkyl, or C₄₋₁₀ alkylcycloalkyl, all optionally substituted with carboxyl, (C₁₋₆ alkoxy)carbonyl, amido, (lower alkyl)amide, or amino optionally mono- or di-substituted with C₁₋₆ alkyl;

Y is H or methyl;

R³ is C₁₋₈ alkyl;

R² is:

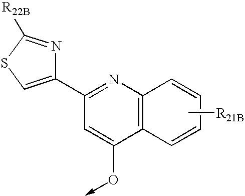

wherein R_{22B} is C₁₋₆ alkyl; amino optionally mono- or di-substituted with C₁₋₆alkyl or C₃₋₇ cycloalkyl; (lower alkyl)amide; or amido; and R_{21B} is C₁₋₆ alkyl, C₁₋₆ alkoxy, amino, di(lower alkyl)amino, (lower alkyl)amide, NO₂, OH, halo, trifluoromethyl, or carboxyl; and

P1 is

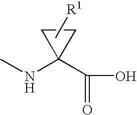

and R¹ is ethyl, vinyl, cyclopropyl, 1 or 2-bromoethyl or 1 or 2-bromovinyl;

Numerous specific compounds that are representative of the compounds of the present invention may be found in Llinas-Brunet et al., U.S. Pat. No. 6,323,180 B1, (referred to hereinafter as “Llinas-Brunet et al.”), which is herein incorporated by reference.

Some specific compounds are also shown in the Table below:

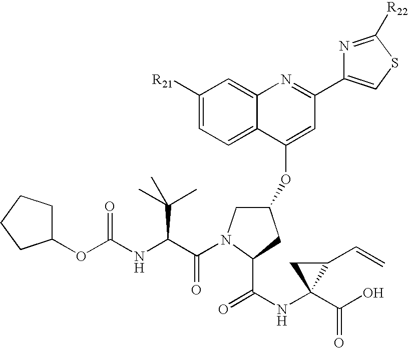

wherein R₂₁ R₂₂ are as defined below:

| Cpd # | R₂₁ | R₂₂ |
|---|---|---|
| 1101 | MeO— | 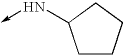 |
| 1102 | MeO— | 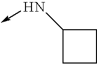 |
| 1103 | MeO— | 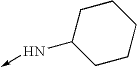 |
| 1104 | MeO— | 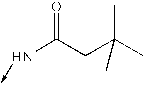 |
| and 1105 | (Me)₂N— | 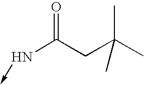 |

The compounds of formula I may be synthesized by the procedures fully set forth in Llinas-Brunet et al. For example, the compounds of formula I may be synthesized according to a general process as illustrated in scheme I (wherein CPG is a carboxyl protecting group and APG is an amino protecting group):

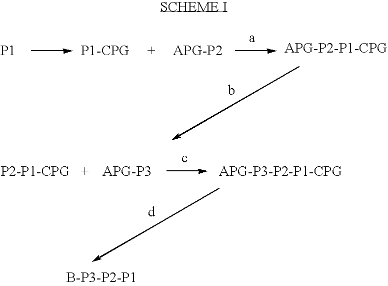

Briefly, the P1, P2, and P3 can be linked by well known peptide coupling techniques. The P1, P2, and P3 groups may be linked together in any order as long as the final compound corresponds to peptides of Formula I. For example, P3 can be linked to P2-P1; or P1 linked to P3-P2. Llinas-Brunet et al. provides numerous examples of preparing various compounds of the formula (I) using this synthetic procedure.

### Methods of Therapeutic Use

The compounds of formula I are effective as HCV protease inhibitors, and these compounds and pharmaceutical compositions comprising these compounds are therefore useful in inhibiting the replication of HCV and in the treatment of HCV infections.

As discussed above, the pharmaceutical compositions of the present invention may be formulated into a variety of dosage forms depending upon the particular composition contemplated. Likewise, a variety of modes of administration are possible depending upon the particular composition and dosage form, although oral administration by tablet, capsule or suspension are the preferred modes of administration.

Dosage levels of the compounds of formula (I) and various treatment regimens in the monotherapy for the prevention and treatment of HCV infection that would be useful are as set forth in Llinas-Brunet et al. As the skilled artisan will appreciate, however, lower dosages may be possible with the compositions of the present invention depending on the level of improvement in bioavailability. Combination therapy is also possible with one or more additional therapeutic or prophylactic agents as fully described by Llinas-Brunet et al. The additional agent(s) may be combined with the compounds of this invention to create a single dosage form or, alternatively, these additional agent(s) may be separately administered to a mammal as part of a multiple dosage form.

In order that this invention be more fully understood, the following examples are set forth. These examples are for the purpose of illustrating embodiments of this invention, and are not to be construed as limiting the scope of the invention in any way.

### EXAMPLES

Formulation #1 (Co-Solvent System)

| Ingredient | Weight (mg/g) | % (w/w) |
|---|---|---|
| Compound of | 40 | 4 |
| formula (I) | | |
| Tromethamine | 32 | 3.2 |
| Water | 448 | 44.8 |
| Ethanol | 213 | 21.3 |
| Propylene glycol | 267 | 26.7 |

Formulation #2 (Co-Solvent System)

| Ingredient | Weight (mg/g) | % (w/w) |
|---|---|---|
| Compound of | 100 | 10 |
| formula (I) | | |
| Tromethamine | 30 | 3 |
| Water | 420 | 42 |
| Ethanol | 200 | 20 |
| Propylene glycol | 250 | 25 |

### Preparation of Formulations 1 and 2

First, Tromethamine is dissolved in a mixture of water, ethanol and propylene glycol in a tightly capped container, and then a Compound of formula (I) is added to the solution and stirring is continued until all the drug becomes soluble.

Formulation #3 (SEDDS)

| Ingredient | Weight (mg/g) | % (w/w) |
|---|---|---|
| Compound of | 40 | 4 |
| formula (I) | | |
| Tromethamine | 8 | 0.8 |
| Ethanol | 94.7 | 9.47 |
| Propylene glycol | 111.5 | 11.15 |
| Water | 16 | 1.6 |
| Propyl gallate | 2 | 0.2 |
| Capmul MCM | 334.4 | 33.44 |
| Cremophor EL | 393.4 | 39.34 |

Formulation #4 (SEDDS)

| Ingredient | Weight (mg/g) | % (w/w) |
|---|---|---|
| Compound of | 125 | 12.5 |
| formula (I) | | |
| Tromethamine | 20 | 2 |
| Ethanol | 50 | 5 |
| Propylene glycol | 50 | 5 |
| Water | 20 | 2 |
| Propyl gallate | 2 | 0.2 |
| PEG3350 | 75 | 7.5 |
| Capmul MCM | 329 | 32.9 |
| V_{E} TPGS | 329 | 32.9 |

Formulation #5 (Lipid-Based System)

| Ingredient | Weight (mg/g) | % (w/w) |
|---|---|---|
| Compound # 1104 | 100 | 10 |
| Tromethamine | 4 | 0.4 |
| Ethanol | 100 | 10 |
| Alpha-Tocopherol | 2 | 0.2 |
| Kollidon 12PF | 50 | 5 |
| Capmul MCM | 744 | 74.4 |

Formulation #6 (SEDDS)

| Ingredient | Weight (mg/g) | % (w/w) |
|---|---|---|
| Compound # 1104 | 100 | 10 |
| Tromethamine | 4 | 0.4 |
| Ethanol | 100 | 10 |
| Propylene glycol | 50 | 5 |
| Alpha-Tocopherol | 2 | 0.2 |
| Kollidon 12PF | 50 | 5 |
| Capmul MCM | 347 | 34.7 |
| V_{E} TPGS | 347 | 34.7 |

Formulation #7 (SEDDS)

| Ingredient | Weight (mg/g) | % (w/w) |
|---|---|---|
| Compound of | 100 | 10 |
| formula (I) | | |
| Tromethamine | 10 | 1 |
| Ethanol | 100 | 10 |
| Propylene glycol | 50 | 5 |
| Water | 20 | 2 |
| Alpha-Tocopherol | 4 | 0.4 |
| Capmul MCM | 220 | 22 |
| V_{E} TPGS | 496 | 49.6 |

Formulation #8 (SEDDS)

| Ingredient | Weight (mg/g) | % (w/w) |
|---|---|---|
| Compound of | 100 | 10 |
| formula (I) | | |
| Tromethamine | 10 | 1 |
| Sodium hydroxide | 3 | 0.3 |
| Ethanol | 100 | 10 |
| Propylene glycol | 50 | 5 |
| Water | 30 | 3 |
| Alpha-Tocopherol | 4 | 0.4 |
| Captex 355 | 220 | 22 |
| V_{E} TPGS | 483 | 48.3 |

### Preparation of Formulations 3, 4, 5, 6, 7 and 8

First, the liquid components such as Capmul MCM or Captex 355, Cremophor EL, propylene glycol, water and ethanol are mixed together in a tightly capped container, and then Tromethamine and antioxidant are dissolved in the mixture. Finally, a Compound of formula (I) is added to the container and stirring is continued until the drug is completely solubilized. When V_{E} TPGS is in the formulation, the mixture is heated at 40° C. in a water bath to melt it before the drug is added. These formulations can be filled into hard shell or soft gelatin capsules.

Formulation #9 (Solid Dispersion —Co-Melt)

| Ingredient | Weight (mg/g) | % (w/w) |
|---|---|---|
| Compound # 1104 | 125 | 12.5 |
| Tromethamine | 20 | 2 |
| PEG1000 | 755 | 75.5 |
| V_{E} TPGS | 100 | 10 |

Formulation #10 (Solid Dispersion —Co-Melt)

| Ingredient | Weight (mg/g) | % (w/w) |
|---|---|---|
| Compound of | 100 | 10 |
| formula (I) | | |
| Tromethamine | 30 | 3 |
| PEG1450 | 770 | 77 |
| V_{E} TPGS | 100 | 10 |

### Preparation of Formulations 9 and 10

PEG and V_{E} TPGS are placed in a tightly capped container and melted at 60° C. in a water bath. Then, Tromethamine and Compound of formula (I) are added to the container and stirring is continued at the same temperature until the drug is completely solubilized. These formulations can be filled into hard shell or soft gelatin capsules.

Formulation #11 (Solid Dispersion - Co-Precipitate -
Comparison Formulation)

| Ingredient | Weight (mg/g) | % (w/w) |
|---|---|---|
| Compound of | 200 | 20 |
| formula (I) | | |
| Kollidon 25 | 800 | 80 |

Formulation #12 (Solid Dispersion - Co-Precipitate -
Invention Formulation)

| Ingredient | Weight (mg/g) | % (w/w) |
|---|---|---|
| Compound of | 300 | 30 |
| formula (I) | | |
| Kollidon 25 | 670 | 67 |
| Tween 80 | 20 | 2 |
| Tromethamine | 10 | 1 |

### Preparation of Formulations 11 and 12

Kollidon 25 and other excipients (e.g., Tween 80 and tromethamine) are dissolved in a sufficient amount of ethanol in a glass container. Then Compound of formula (I) is added to the container and stirred until the compound is completely dissolved. The ethanol is removed by placing the container in a vacuum oven at RT. After the ethanol is completely evaporated, the solid material (co-precipitate) is taken out from the glass container and passed through a 1-mm screen. The powder can be filled into hard shell capsules or further compressed into tablets. The solvent used to dissolve the drug and the excipients can be ethanol, methanol, or chloroform.

Formulation #13 (Dry Granulation)

| Ingredient | Weight (mg/g) | % (w/w) |
|---|---|---|
| Compound of | 225 | 22.5 |
| formula (I) | | |
| Lactose | 675 | 67.5 |
| Tromethamine | 67.5 | 6.75 |
| SLS | 22.5 | 2.25 |
| Mg Stearate | 10 | 1.0 |

Formulation #14 (Dry Granulation)

| Ingredient | Weight (mg/g) | % (w/w) |
|---|---|---|
| Compound of | 225 | 22.5 |
| formula (I) | | |
| PEG 4600 | 675 | 67.5 |
| Tromethamine | 67.5 | 6.75 |
| SLS | 22.5 | 2.25 |
| Mg Stearate | 10 | 1.0 |

### Preparation of Formulations #13 and #14

In a glass mortar, the formulation ingredients are triturated for about 2 minutes with a glass pestle. The mixture is transferred into a glass bottle and blended with a torbola blender for 6 minutes. The magnesium stearate is added to the powder and blending is continued for another 4 minutes. The powder can be compressed into tablets @ 6.6KN using a 11 mm die set.

Formulation #15 (Wet Granulation)

| Ingredient | Weight (mg/g) | % (w/w) |
|---|---|---|
| Compound of | 238 | 23.8 |
| formula (I) | | |
| Lactose | 714 | 71.4 |
| PVP (5%) | 48 | 4.8 |

Formulation #16 (Wet Granulation)

| Ingredient | Weight (mg/g) | % (w/w) |
|---|---|---|
| Compound of | 230 | 23 |
| formula (I) | | |
| Lactose | 688 | 68.8 |
| Tromethamine | 34 | 3.4 |
| PVP (5%) | 48 | 4.8 |

Formulation #17 (Wet Granulation)

| Ingredient | Weight (mg/g) | % (w/w) |
|---|---|---|
| Compound of | 216 | 21.6 |
| formula (I) | | |
| PEG 4600 | 649 | 64.9 |
| Tromethamine | 65 | 6.5 |
| SLS | 22 | 2.2 |
| PVP (5%) | 48 | 4.8 |

### Preparation of Formulations # 15, 16 and 17

In a glass mortar, the formulation ingredients are triturated for about 2 minutes with the glass pestle. Hot water (80° C.) is added dropwise to the mixture while stirring with the pestle. Water addition is continued until a paste is obtained. The paste is dried in a petridish in an oven at 45° C. After 2 hours drying, the paste is triturated and passed through a mesh # 18. The powder is dried until the weight is constant and equal to the initial weight. The powder can be filled into hard shell capsules or compressed into tablets.

### In-Vitro Dispersion and Dissolution Studies

(1) Dispersion Test

To assess the dispersability, each prepared formulation may be diluted with pH 2.0 (0.05M HCl/KCl) and pH 6.8 buffer (0.05M KH₂PO₄/K₂HPO), the dispersion is observed as clear solution, colloidal dispersion (emulsion or microemulsion) or suspension with drug precipitation. Formulations with no drug precipitation in the buffers and faster dispersion rate are preferred.

(2) Dissolution Test

USP XXIII apparatus (paddle method, 50 rpm) may be used to obtain the release of drug from selected formulations into 900 ml pH 2.0 buffer (0.05M HCl/KCl) dissolution medium at 37° C. Samples of 10 ml are withdrawn at various time intervals and drug concentration is determined by HPLC. Formulations with faster and higher drug release are preferred.

We claim:

1. A pharmaceutical composition comprising:
(a) a compound having the following formula (I): 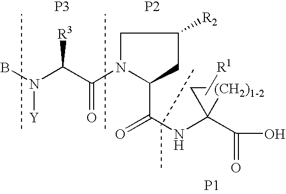
wherein B is H, a C₆ or C₁₀ aryl, C₇₋₁₆ aralkyl; Het or (lower alkyl)-Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide;
or B is an acyl derivative of formula R₄—C(O)—; a carboxyl derivative formula R₄—O—C(O)—; an amide derivative of formula R₄—N(R₅)—C(O)—; a thioamide derivative of formula R₄—N(R₅)—C(S)—; or a sulfonyl derivative of formula R₄—SO₂ wherein
R₄ is (i) C₁₋₁₀ to alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;
(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;
(iii) amino optionally mono- or di-substituted with C₁₋₆ alkyl; amido; or (lower alkyl)amide;
(iv) C₆ or C₁₀ aryl or C₇₋₁₆ aralkyl, all optionally substituted with C₁₋₆ alkyl, hydroxy, amido, (lower alkyl)amide, or amino optionally mono- or di-substituted with C₁₋₆ alkyl; or
(v) Het or (lower alkyl)-Het, both optionally substituted with C₁₋₆ alkyl, hydroxy, amido, (lower alkyl) amide, or amino optionally mono- or di-substituted with C₁₋₆ alkyl;
R₅ is H or C₁₋₆ alkyl;
with the proviso that when B is a carboxyl derivative, an amide derivative or a thioamide derivative, R₄ is not a cycloalkoxy;
Y is H or C₁₋₆ alkyl;
R³ is C₁₋₈ alkyl, C₃₋₇ cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;
R² is CH₂—R₂₀, NH—R₂₀, O—R₂₀ or S—R₂₀, wherein R₂₀ is quinolyl or (lower alkyl)quinolyl, both optionally mono-, di- or tri-substituted with R₂₁,
wherein each R₂₁ is independently C₁₋₆ alkyl; C₁₋₆ alkoxy; lower thioalkyl; sulfonyl; NO₂; OH; SH; halo; haloalkyl; amino optionally mono- or di-substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;
wherein R₂₂ is C₁₋₆ alkyl; C₃₋₇ cycloalkyl; C₁₋₆ alkoxy; amino optionally mono- or di-substituted with C₁₋₆ alkyl or C₃₋₇cycloalkyl; sulfonyl; (lower alkyl)sulfonyl; NO₂; OH; SH; halo; haloalkyl; carboxyl; amide; (lower alkyl)amide; or Het optionally substituted with C₁₋₆ alkyl;
R¹ is H; C₁₋₆ alkyl, C₃₋₇ cycloalkyl, C₂₋₆ alkenyl, or C₂₋₆ alkynyl, all optionally substituted with halogen;
or a tautomer thereof;
(b) about 0.1 to 10% by weight of a pharmaceutically acceptable amine or a mixture of pharmaceutically acceptable amines; and
(c) one or more pharmaceutically acceptable oils, carriers or hydrophilic solvents;
and when (c) is one or more pharmaceutically acceptable oils, the pharmaceutical composition further comprises:
(d) optionally one or more pharmaceutically acceptable hydrophilic solvents;
(e) optionally one or more pharmaceutically acceptable polymers; and
(f) optionally one or more pharmaceutically acceptable surfactants;
and when (c) is one or more pharmaceutically acceptable carriers, the pharmaceutical composition further comprises:
(d) optionally one or more pharmaceutically acceptable surfactants.

2. A pharmaceutical composition according to claim 1, wherein the compound of formula (I) is present in an amount of from about 1% to 50% by weight.

3. A pharmaceutical composition according to claim 1, wherein the amine is present in an amount of from about 0.1% to 7% by weight.

4. A pharmaceutical composition according to claim 1, wherein the amine is a C₁₋₆ alkylamine, di-(C₁₋₆ alkyl)-amine or tri-(C₁₋₆ alkyl)-amine, wherein one or more alkyl groups thereof may be optionally substituted by one or more hydroxy groups, or the amine is C₁₋₆ alkylenediamine, a basic amino acid or choline hydroxide, or mixtures thereof.

5. A pharmaceutical composition according to to claim 1, wherein the amine is selected from ethanolamine, diethanolamine, triethanolamine, tris(hydroxymethyl)aminomethane, ethylenediamine or dimethylaminoethanol, or mixtures thereof.

6. A pharmaceutical composition according to to claim 1, wherein the one or more pharmaceutically acceptable oils, carriers or hydrophilic solvents are present in an amount of from about 1% to 99% by weight.

7. A pharmaceutical composition according to claim 1, wherein the pharmaceutically acceptable oil is selected from: medium or long chain mono-, di- or triglycerides, water insoluble vitamins, fatty acids and mixtures thereof.

8. A pharmaceutical composition according to claim 1, wherein the pharmaceutically acceptable oil is selected from: mono-, di- or triglycerides of caprylic fatty acids; mono-, di- or triglycerides of capric fatty acids; oleic acid, and mixtures thereof.

9. A pharmaceutical composition according to claim 1, wherein the pharmaceutically acceptable carrier is selected from a pharmaceutically acceptable polymer and a pharmaceutically acceptable urea.

10. A pharmaceutical composition according to claim 1, wherein the pharmaceutically acceptable carrier is selected from polyethylene glycols, polyvinylpyrrolidones, polyvinylalcohols, cellulose derivatives, polyacrylates, polymethacrylates, polyglycolyzed glycerides, ureas, sugars, polyols, and mixtures thereof.

11. A pharmaceutical composition according to claim 1, wherein the pharmaceutically acceptable hydrophilic solvent is selected from propylene glycol, polypropylene glycol, polyethylene glycol, glycerol, ethanol, dimethyl isosorbide, glycofurol, propylene carbonate, dimethyl acetamide, water, or mixtures thereof.

12. A pharmaceutical composition according to claim 1, wherein the pharmaceutically acceptable hydrophilic solvent is selected from propylene glycol, polyethylene glycol, ethanol, water, and mixtures thereof.

13. A pharmaceutical composition according to claim 1, wherein the pharmaceutically acceptable polymer is present in an amount of up to about 50% by weight.

14. A pharmaceutical composition according to claim 1, wherein the pharmaceutically acceptable polymer is selected from polyethylene glycols, polyvinylpyrrolidones, polyvinylalcohols, cellulose derivatives, polyacrylates, polymethacrylates, sugars, polyols, and mixtures thereof.

15. A pharmaceutical composition according to claim 1, wherein the pharmaceutically acceptable surfactant is present in an amount of up to about 70% by weight.

16. A pharmaceutical composition according to claim 1, wherein the pharmaceutically acceptable surfactant is selected from d-alpha tocopheryl polyethylene glycol 1000 succinate, polyoxyl castor oils, polysorbates, peglicol 6-oleate, polyoxyethylene stearates, polyglycolyzed glycerides or poloxamers, or sodium lauryl sulfate and mixtures thereof.

17. A pharmaceutical composition according to claim 1, wherein the pharmaceutically acceptable surfactant is selected from d-alpha tocopheryl polyethylene glycol 1000 succinate, polyoxyl 40 hydrogenated castor oil, polyoxyl 35 castor oil, polyoxypropylene-polyoxyethylene block copolymer, or sodium lauryl sulfate, and mixtures thereof.

18. A pharmaceutical composition according to claim 1, further comprising one or more pharmaceutically acceptable bases.

19. A pharmaceutical composition according to claim 18, wherein the pharmaceutically acceptable bases is selected from sodium hydroxide, potassium hydroxide, sodium hydrogen carbonate, aluminum hydroxide, magnesium hydroxide, and magnesium aluminum hydroxide.

20. A pharmaceutical composition according to claim 1, wherein in the compound of formula (I):
B is a carboxyl derivative of formula R₄—O—C(O)—, wherein R₄ is
C₃₋₇ cycloalkyl, or C₄₋₁₀ alkylcycloalkyl, all optionally substituted with carboxyl, (C₁₋₆ alkoxy)carbonyl, amido, (lower alkyl)amide, or amino optionally mono- or di-substituted with C₁₋₆ alkyl;
Y is H or methyl;
R³ is C₁₋₈ alkyl;
R₂ is: 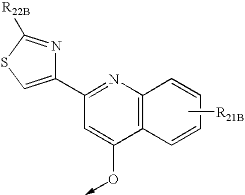
wherein R_{22B} is C₁₋₆ alkyl; amino optionally mono- or di-substituted with C₁₋₆alkyl or C₃₋₇ cycloalkyl; (lower alkyl)amide; or amido; and R_{21B} is C₁₋₆ alkyl, C₁₋₆ alkoxy, amino, di(lower alkyl)amino, (lower alkyl)amide, NO₂, OH, halo, trifluoromethyl, or carboxyl; and
P1 is 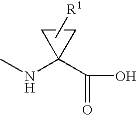
and R¹ is ethyl, vinyl, cyclopropyl, 1 or 2-bromoethyl or 1 or 2-bromovinyl.

21. A pharmaceutical composition according to claim 1, comprising:
(a) a compound of formula (I);
(b) about 0.1 to 10% by weight of a pharmaceutically acceptable amine or a mixture of pharmaceutically acceptable amines; and
(c) one or more pharmaceutically acceptable hydrophilic solvents.

22. A pharmaceutical composition according to claim 1, comprising:
(a) about 5% to 30% by weight of a compound of formula (I);
(b) about 0.5% to 7% by weight of a pharmaceutically acceptable amine; and
(c) about 40% to 99% by weight of pharmaceutically acceptable hydrophilic solvent.

23. A pharmaceutical composition according to claim 1, comprising:
(a) about 5% to 15% by weight of a compound of formula (I);
(b) about 0.5% to 5% by weight of a pharmaceutically acceptable amine; and
(c) about 80% to 99% by weight of pharmaceutically acceptable hydrophilic solvent.

24. A pharmaceutical composition according to claim 1, comprising:
(a) about 5% to 15% by weight of a compound of formula (I);
(b) about 0.5% to 5% by weight of tris(hydroxymethyl)aminomethane; and
(c) about 80% to 90% by weight of a mixture of propylene glycol, ethanol and water.

25. A pharmaceutical composition according to claim 24, wherein in the compound of formula (I):
B is a carboxyl derivative of formula R₄—O—C(O)—, wherein R₄ is
C₃₋₇ cycloalkyl, or C₄₋₁₀ alkylcycloalkyl, all optionally substituted with carboxyl, (C₁₋₆ alkoxy)carbonyl, amido, (lower alkyl)amide, or amino optionally mono- or di-substituted with C₁₋₆ alkyl;
Y is H or methyl;
R³ is C₁₋₈ alkyl;
R² is: 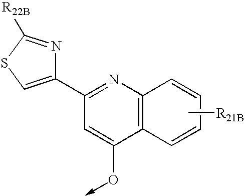
wherein R_{22B} is C₁₋₆ alkyl; amino optionally mono- or di-substituted with C₁₋₆alkyl or C₃₋₇ cycloalkyl; (lower alkyl)amide; or amido; and R_{21B} is C₁₋₆ alkyl, C₁₋₆ alkoxy, amino, di(lower alkyl)amino, (lower alkyl)amide, NO₂, OH, halo, trifluoromethyl, or carboxyl; and
P1 is 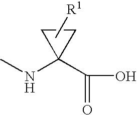
and R¹ is ethyl, vinyl, cyclopropyl, 1 or 2-bromoethyl or 1 or 2-bromovinyl.

26. A pharmaceutical composition according to claim 1, comprising:
(a) a compound of formula (I);
(b) about 0.1 to 10% by weight of a pharmaceutically acceptable amine or a mixture of pharmaceutically acceptable amines;
(c) one or more pharmaceutically acceptable oils;
(d) optionally one or more pharmaceutically acceptable hydrophilic solvents;
(e) optionally one or more pharmaceutically acceptable polymers; and
(f) optionally one or more pharmaceutically acceptable surfactants.

27. A pharmaceutical composition according to claim 1, comprising:
(a) about 5% to 30% by weight of a compound of formula (I);
(b) about 0.1% to 7% by weight of a pharmaceutically acceptable amine;
(c) about 1% to 99% by weight of a pharmaceutically acceptable oil;
(d) up to about 70% by weight of a pharmaceutically acceptable hydrophilic solvent;
(e) optionally up to about 50% by weight of a pharmaceutically acceptable polymer;
(f) up to about 70% by weight of a pharmaceutically acceptable surfactant;
and
(g) optionally about 0.1 to 10% by weight of a pharmaceutically acceptable base.

28. A pharmaceutical composition according to claim 1, comprising:
(a) about 10% to 20% by weight of a compound of formula (I);
(b) about 0.1% to 5% by weight of a pharmaceutically acceptable amine;
(c) about 20% to 70% by weight of a pharmaceutically acceptable oil;
(d) about 10% to 30% by weight of a pharmaceutically acceptable hydrophilic solvent;
(e) optionally about 1% to 20% by weight of a pharmaceutically acceptable polymer;
(f) about 20% to 50% by weight of a pharmaceutically acceptable surfactant; and
(g) optionally about 0.1 to 5% by weight of a pharmaceutically acceptable base.

29. A pharmaceutical composition according to claim 1, comprising:
(a) about 10% to 20% by weight of a compound of formula (I);
(b) about 0.1% to 5% by weight of tris(hydroxymethyl)aminomethane;
(c) about 20% to 70% by weight of a mono- or diglyceride of caprylic fatty acid or a mono- or diglyceride of capric fatty acid, or mixtures thereof;
(d) about 10% to 30% by weight of a mixture of propylene glycol, ethanol and optionally water;
(e) optionally about 1% to 20% by weight of polyethylene glycol or polyvinylpyrrolidone; and
(f) about 20% to 50% by weight of d-alpha tocopheryl polyethylene glycol 1000 succinate or polyoxyl 35 castor oil.

30. A pharmaceutical composition according to claim 29, wherein in the compound of formula (I):
B is a carboxyl derivative of formula R₄—O—C(O)—, wherein R₄ is
C₃₋₇ cycloalkyl, or C₄₋₁₀ alkylcycloalkyl, all optionally substituted with carboxyl, (C₁₋₆ alkoxy)carbonyl, amido, (lower alkyl)amide, or amino optionally mono- or di-substituted with C₁₋₆ alkyl;
Y is H or methyl;
R³ is C₁₋₈ alkyl;
R² is: 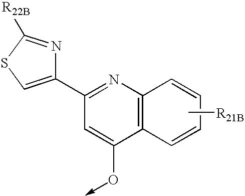
wherein R_{22B} is C₁₋₆ alkyl; amino optionally mono- or di-substituted with C₁₋₆alkyl or C₃₋₇ cycloalkyl; (lower alkyl)amide; or amido; and R_{21B} is C₁₋₆ alkyl, C₁₋₆ alkoxy, amino, di(lower alkyl)amino, (lower alkyl)amide, NO₂, OH, halo, trifluoromethyl, or carboxyl; and
P1 is 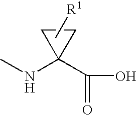
and R¹ is ethyl, vinyl, cyclopropyl, 1 or 2-bromoethyl or 1 or 2-bromovinyl.

31. A pharmaceutical composition according to claim 29, said composition further comprising about 0.1% to 5% by weight of sodium hydroxide.

32. A pharmaceutical composition according to claim 1, comprising:
(a) a compound of formula (I);
(b) about 0.1 to 10% by weight of a pharmaceutically acceptable amine or a mixture of pharmaceutically acceptable amines;
(c) one or more pharmaceutically acceptable carriers; and
(d) optionally one or more pharmaceutically acceptable surfactants.

33. A pharmaceutical composition according to claim 1, comprising:
(a) about 5% to 30% by weight of a compound of formula (I);
(b) about 0.1% to 7% by weight of a pharmaceutically acceptable amine;
(c) about 1% to 99% by weight of a pharmaceutically acceptable carrier; and
(d) up to about 50% by weight of a pharmaceutically acceptable surfactant.

34. A pharmaceutical composition according to claim 1, comprising:
(a) about 10% to 20% by weight of a compound of formula (I);
(b) about 0. 1% to 5% by weight of a pharmaceutically acceptable amine;
(c) about 60% to 80% by weight of a pharmaceutically acceptable carrier; and
(d) about 1% to 20% by weight of a pharmaceutically acceptable surfactant.

35. A pharmaceutical composition according to claim 1, comprising:
(a) about 10% to 20% by weight of a compound of formula (I);
(b) about 0.1% to 5% by weight of tris(hydroxymethyl)aminomethane;
(c) about 60% to 80% by weight of polyethylene glycol, polyvinylpyrrolidone, lactose or a mixture thereof; and
(d) about 1% to 20% by weight of d-alpha tocopheryl polyethylene glycol 1000 succinate, polyoxypropylene-polyoxyethylene block copolymer or sodium lauryl sulfate.

36. A pharmaceutical composition according to claim 35, wherein in the compound of formula (I):
B is a carboxyl derivative of formula R₄—O—C(O)—, wherein R₄ is
C₃₋₇ cycloalkyl, or C₄₋₁₀ alkylcycloalkyl, all optionally substituted with carboxyl, (C₁₋₆ alkoxy)carbonyl, amido, (lower alkyl)amide, or amino optionally mono- or di-substituted with C₁₋₆ alkyl;
Y is H or methyl;
R³ is C₁₋₈ alkyl;
R² is: 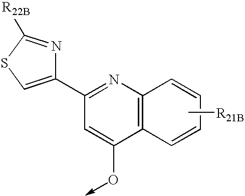
wherein R_{22B} is C₁₋₆ alkyl; amino optionally mono- or di-substituted with C₁₋₆alkyl or C₃₋₇ cycloalkyl; (lower alkyl)amide; or amido; and R_{21B} is C₁₋₆ alkyl, C₁₋₆ alkoxy, amino, di(lower alkyl)amino, (lower alkyl)amide, NO₂, OH, halo, trifluoromethyl, or carboxyl; and
P1 is 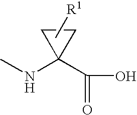
and R¹ is ethyl, vinyl, cyclopropyl, 1 or 2-bromoethyl or 1 or 2-bromovinyl;

37. A pharmaceutical composition according to claim 1, in the form of a fluid dosage form selected from a hard shell or softgel capsule or in the form of a solid dosage form selected from a powder, a tablet or a capsule.

38. A pharmaceutical composition according to claim 1, further comprising one or more antioxidants.

39. A pharmaceutical composition according to claim 38, wherein the antioxidant is present in an amount of about 0.01 to 1% by weight.

40. A pharmaceutical composition according to claim 38, wherein the antioxidant is selected from ascorbic acid, sulfatide salts, citric acid, propyl gallate, dl-a-tocopherol, ascorbyl palmitate, BHT or BHA.

41. A method of manufacturing a pharmaceutical composition according to claim 1, said method comprising:
(A) (a) dissolving the amine(s) in the one or more pharmaceutically acceptable solvents; (b) adding the compound of formula (I) to the solution obtained in step (a) and mixing; or
(B) (a) mixing together the pharmaceutically acceptable oil(s), surfactant(s) and solvent(s); (b) dissolving the pharmaceutically acceptable amine(s) in the mixture obtained in step (a); (c) optionally heating the mixture obtained in step (b) if necessary to sufficiently melt one or more of the components of the mixture; (d) adding the compound of formula (I) to the mixture obtained in steps (b) or (c) and mixing; or
(C) (a) dissolving the pharmaceutically acceptable amine(s), the pharmaceutically acceptable carrier(s) and optionally the pharmaceutically acceptable surfactant(s) in a suitable hydrophilic solvent; (b) adding the compound of formula (I) to the solution obtained in step (a) and mixing to dissolve the compound of formula (I); (c) evaporating the hydrophilic solvent to cause co-precipitation of the compound of formula (I), the amine(s), the carrier(s) and the optional surfactant(s); or
(D) (a) mixing the pharmaceutically acceptable carrier(s) and the optional surfactant(s) and heating the resulting mixture to sufficiently melt the carrier(s) and surfactant(s); (b) adding the pharmaceutically acceptable amine(s) and the compound of formula (I) to the mixture obtained in step (a) and mixing; or
(E) (a) mixing the compound of formula (I), the pharmaceutically acceptable amine(s), the pharmaceutically acceptable carrier(s) and optionally the pharmaceutically acceptable surfactant(s) to form a blend, and (b) optionally adding a lubricant to the blend; or
(F) (a) mixing the compound of formula (I), the pharmaceutically acceptable amine(s), the pharmaceutically acceptable carrier(s) and optionally the pharmaceutically acceptable surfactant(s) while adding water or another hydrophilic solvent(s) to the mixture to obtain a paste; (b) drying the paste of step (a) to a sufficient level of dryness; and (c) passing the dried paste through a screen.

42. A method of inhibiting the replication of hepatitis C virus by exposing the virus to a hepatitis C viral NS3 protease inhibiting amount of the composition according to claim 1.

43. A method of treating a hepatitis C viral infection in a mammal comprising administering to a mammal in need thereof a therapeutically effective amount of the composition according to claim 1.
